(19) United States
(12) Patent Application Publication
(10) Pub. No.: US 2022/0168016 A1
(43) Pub. Date: Jun. 2, 2022

(54) FIXING SUPPORT TOOL OF BALLOON UNIT FOR UTERINE HEMOSTASIS

(71) Applicants: ATOM MEDICAL CORPORATION, Bunkyo-ku, Tokyo (JP); KYOTO UNIVERSITY, Kyoto-shi, Kyoto (JP)

(72) Inventors: Masato HONDA, Saitama City, Saitama (JP); Shinichi KOBAYASHI, Saitama City, Saitama (JP); Ryoko KAKINUMA, Saitama City, Saitama (JP); Ichiro MATSUBARA, Bunkyo-ku, Tokyo (JP); Eiji KONDOH, Kyoto-shi, Kyoto (JP)

(21) Appl. No.: 17/601,007

(22) PCT Filed: Mar. 11, 2020
(86) PCT No.: PCT/JP2020/010483

§ 371 Date: Oct. 1, 2021

(30) Foreign Application Priority Data
Apr. 10, 2019 (JP) 2019-074613

(51) Int. Cl. A61B 17/42

(52) U.S. Cl. CPC A61B 17/42; A61B 2017/00557; A61B 2017/4216

(57) ABSTRACT

Provided is a fixing support tool of a balloon unit for uterine hemostasis, which can prevent an inflated balloon of the balloon unit for uterine hemostasis from falling out of a uterus. A fixing support tool 100 is used to fix a balloon unit for uterine hemostasis 1 including a flexible tube and a hemostatic balloon provided in a distal end of the tube to a patient M. The fixing support tool 100 has a tube stopper 7 that supports the tube, and a fixing member 8 capable of fixing the tube stopper to the patient.

### TECHNICAL FIELD

The present invention relates to a fixing support tool of a balloon unit for uterine hemostasis that suppresses or stops uterine bleeding.

Priority is claimed on Japanese Patent Application No. 2019-74613, filed on Apr. 10, 2019, the content of which is incorporated herein by reference.

### BACKGROUND ART

In the related art, a Balloon Tamponade (BT method) is known as follows. A balloon for hemostasis is inserted from a vagina, and is disposed inside a uterus. Thereafter, a liquid is injected into the balloon to inflate the balloon. In this manner, uterine contraction is strengthened to suppress bleeding. The balloon unit for hemostasis used for the BT method is provided a drain flow path for discharging blood or the like inside the uterus, and a supply-discharge flow path for injecting or discharging the liquid into or from the balloon. Normally, when the balloon is inserted into the uterus from an outside of body, the balloon is inserted into the vagina, and thereafter, the balloon is pushed into the uterus to be disposed inside the uterus. Then, the balloon is inflated by injecting the liquid into the balloon via a connector of the supply-discharge flow path provided in an end portion on a side opposite to the balloon.

For example, as a balloon unit for hemostasis having one balloon, a balloon unit for hemostasis disclosed in PTL 1 is known. The balloon unit for hemostasis disclosed in PTL 1 is configured to include a balloon catheter in which a balloon for hemostasis is provided in a distal end portion of a flexible tube and a connector is provided in a proximal end portion of the tube, and a connector cover detachably attached to a proximal end portion of the balloon catheter. The balloon unit is inserted into the uterus in a state where a first terminal and a second terminal are gathered by the connector cover.

On the other hand, for example, as a balloon unit for uterine hemostasis provided with two balloons, a balloon tube instrument disclosed in PTL 2 is known. The balloon tube instrument disclosed in PTL 2 includes a tube shaft including a passage through which one end side communicates with the other end side, a first balloon provided in an intermediate portion of the tube shaft, a second balloon disposed adjacent to the first balloon, a fluid route for injecting and discharging a fluid into and from the first balloon and the second balloon, and a fluid injection port and a fluid discharge port which are provided in both end portions of the fluid route. The first balloon is disposed inside the uterus, and the second balloon is disposed in a state of being inflated inside the vagina. In this manner, the first balloon inside the uterus can be disposed in a state where the first balloon is reliably brought into close contact with a bleeding site (site on a uterine os side inside the uterus) in a case of placenta previa.

### CITATION LIST

### Patent Literature

[PTL 1] Japanese Patent No. 6012428
[PTL 2] JP-A-2016-221247

### SUMMARY OF INVENTION

### Technical Problem

In many cases of hemostatic treatment using a uterine balloon unit in the related art as described above, failures occur due to the balloon slipping out of the uterine os. When the treatment fails, a hysterectomy has to be performed in many cases. Therefore, it is necessary to prevent the inflated balloon from falling out of the uterus in order to save a patient's life and ensure quality of life (QOL). Currently, the balloon is prevented from falling out of the uterus as follows. An inside of the vagina is filled with gauze, or a vaginal portion of cervix is gripped with cervical laceration forceps. In addition, a doctor or a nurse continuously grips a tube shaft (tube).

However, even when the inside of the vagina is filled with the gauze to prevent the balloon from falling out, a contractile force of the uterus is maintained in many cases. Accordingly, the balloon easily slips off, and there is a risk in that the gauze may be unintentionally left behind inside vagina. In addition, in a method of gripping the vaginal portion of cervix with the cervical laceration forceps, there is a possibility that the cervical laceration forceps may injure the vaginal portion of cervix. Furthermore, when the doctor or the nurse continuously grip the tube, not only a considerable physical burden is imposed on the doctor or the nurse, but also the doctor or the nurse has to perform one-handed treatment. Consequently, another treatment cannot be performed in some cases.

The present invention is made in view of the above-described circumstances, and an object thereof is to provide a fixing support tool of a balloon unit for uterine hemostasis which can prevent an inflated balloon of the balloon unit from falling out of a uterus.

### Solution to Problem

According to the present invention, there is provided a fixing support tool of a balloon unit for uterine hemostasis, which is used to fix a balloon unit for uterine hemostasis including a flexible tube and a hemostatic balloon provided in a distal end of the tube to a patient. The fixing support tool has a tube stopper that supports the tube, and a fixing member capable of fixing the tube stopper to the patient.

In the present invention, the tube stopper is fixed to the patient by the fixing member. Accordingly, the tube supported by the tube stopper is fixed to the patient. Therefore, the balloon inflated inside a uterus can be prevented from falling out of the uterus.

As a preferable aspect of the fixing support tool of a balloon unit for uterine hemostasis according to the present invention, the tube stopper may have a pinching portion that pinches the tube.

In the above-described aspect, the tube is pinched and supported by the pinching portion of the tube stopper. Accordingly, the tube stopper can be reliably supported in the tube, and the tube stopper is fixed to the patient. In this manner, the balloon inflated inside the uterus can be further prevented from falling out of the uterus.

As an aspect of the fixing support tool of a balloon unit for uterine hemostasis according to the present invention, the fixing member may have an abdominal belt attachable to an abdomen of the patient, and a band connecting the abdominal belt and the tube stopper to each other.

In the above-described aspect, the fixing member is configured to include the abdominal belt and the band. Accordingly, the tube stopper can be reliably fixed.

As another aspect of the fixing support tool of a balloon unit for uterine hemostasis according to the present invention, the fixing support tool may have a tubular tube cover that covers the tube. The tube stopper may support the tube via the tube cover.

Here, when rigidity of the tube of the balloon unit for uterine hemostasis is excessively low (excessively soft), even if the tube is pinched by the tube stopper and the tube stopper is fixed to the patient, a possibility that the tube may not be properly supported is considered. In addition, similarly, when the tube is excessively thin, a possibility that the tube may not be properly supported by the tube stopper is considered.

In contrast, in the above-described aspect, the tube cover is attached to the tube. Accordingly, it is possible to improve the rigidity of the tube (rigidity as a composite structure in which the tube and the tube cover are combined with each other). In addition, an outer diameter of the tube when the tube cover is attached to the tube (outer diameter of the composite structure in which the tube and the tube cover are combined with each other) is larger than an outer diameter of the tube when the tube is a single body. Accordingly, the tube can be properly supported by the tube stopper.

The tube cover may not necessarily have the rigidity higher than that of the tube. As long as the rigidity as the composite structure in which the tube and the tube cover are combined with each other is improved to some extent, the tube cover may have the rigidity the same as that of the tube. Alternatively, the tube cover may have the rigidity lower than that of the tube.

As still another aspect of the fixing support tool of a balloon unit for uterine hemostasis according to the present invention, the tube cover may have a slit extending in a length direction of the tube cover, and opening the tube cover so that the tube cover is attached to the tube.

In the above-described aspect, the tube cover has the slit. Accordingly, the tube cover can be easily attached to the tube.

As still another aspect of the fixing support tool of a balloon unit for uterine hemostasis according to the present invention, the tube cover may have a plurality of fixing portions for fixing the tube stopper at different positions in a length direction of the tube.

In the above-described aspect, the tube cover is provided with the plurality of fixing portions. Accordingly, the tube stopper can be fixed at a desired position in the length direction of the tube. Moreover, the tube stopper pinching the tube is prevented from sliding and moving along the tube cover even when an unexpected situation occurs. In this manner, a pinching position pinched by the tube stopper can be maintained.

As still another aspect of the fixing support tool of a balloon unit for uterine hemostasis according to the present invention, the fixing portion may include a recess portion recessed toward a center of the tube cover from an outer peripheral surface of the tube cover.

In the above-described aspect, the tube stopper pinches any of the plurality of recess portion. Accordingly, the tube stopper is prevented from sliding and moving along the tube cover. In this manner, the pinching position pinched by the tube stopper can be reliably maintained. In addition, the plurality of fixing portions do not project outward of the outer peripheral surface of the tube cover. Accordingly, when the tube having the attached tube cover is inserted into the vagina of a mother's body, the tube can be smoothly inserted into the vagina, and the burden on the mother's body can be reduced.

As still another aspect of the fixing support tool of a unit for uterine hemostasis according to the present invention, the fixing portion may include a projection portion projecting from an outer peripheral surface of the tube cover.

In the above-described aspect, the tube stopper is locked by the plurality of projection portions projecting from the outer peripheral surface of the tube cover. Accordingly, the tube stopper is prevented from sliding and moving along the tube cover. In this manner, the pinching position pinched by the tube stopper can be reliably maintained. In addition, each of the plurality of fixing portions includes the projection portion. Accordingly, the rigidity of the tube cover can be improved.

As a preferable aspect of the fixing support tool of a unit for uterine hemostasis according to the present invention, a cushion portion may be formed in an end portion of the tube cover on the hemostatic balloon side when the tube cover is attached to the tube.

In the above-described aspect, even when the tube cover is pushed toward the inflated balloon in a state where the tube cover is attached to the tube, the cushion portion is provided in a distal end portion of the tube cover. Accordingly, the hemostatic balloon can be prevented from being damaged by the tube cover.

### Advantageous Effects of Invention

According to the present invention, the inflated balloon can be prevented from falling out of the uterus.

### BRIEF DESCRIPTION OF DRAWINGS

FIG. 1 is a plan view of a balloon unit for uterine hemostasis supported by a fixing support tool of the balloon unit for uterine hemostasis according to a first embodiment of the present invention.

FIG. 2 is a front view when a second balloon of the balloon unit for uterine hemostasis according to the first embodiment is viewed from a distal end side.

FIG. 3 is a cross-sectional view taken along arrow line A1-A1 illustrated in FIG. 1 of the second balloon in the first embodiment.

FIG. 4 is a cross-sectional view taken along arrow line B1-B1 illustrated in FIG. 1 of a first tube in which a second tube according to the first embodiment is accommodated.

FIG. 5 is a longitudinal sectional view of a first balloon in which the second balloon according to the first embodiment is accommodated.

FIG. 6 is a view illustrating a state where the balloon unit for uterine hemostasis according to the first embodiment is inserted into a uterus and is supported by the fixing support tool.

FIG. 7 is a perspective view illustrating a tube stopper forming the fixing support tool of the balloon unit for uterine hemostasis according to the first embodiment.

FIG. 8 is a schematic side view illustrating a state where the balloon unit for uterine hemostasis according to the first embodiment is disposed inside the uterus and is supported by the fixing support tool.

FIG. 9 is a view illustrating a state where a tube cover of a fixing support tool is attached to a balloon unit for uterine hemostasis according to a second embodiment of the present invention.

FIG. 10 is a side view of the tube cover forming the fixing support tool according to the second embodiment.

FIG. 11 is a perspective view illustrating a state where the tube stopper of the fixing support tool according to the second embodiment supports a tube of the balloon unit for uterine hemostasis via the tube cover.

FIG. 12 is a schematic side view illustrating a state where the balloon unit for uterine hemostasis according to the second embodiment is disposed inside the uterus and is supported by the fixing support tool.

FIG. 13 is a perspective view illustrating a tube cover according to a first modification example of the second embodiment.

FIG. 14 is a perspective view illustrating a state where a tube stopper of a fixing support tool according to the first modification example supports a tube of the balloon unit for uterine hemostasis via the tube cover.

FIG. 15 is a schematic side view illustrating a state where the balloon unit for uterine hemostasis having the attached tube cover according to the first modification example is disposed inside the uterus and is supported by the fixing support tool.

FIG. 16 is a cross-sectional view illustrating a cross section of a tube cover according to a second modification example of the second embodiment.

### DESCRIPTION OF EMBODIMENTS

### First Embodiment

Hereinafter, a first embodiment of a fixing support tool of a balloon unit for uterine hemostasis according to the present invention (hereinafter, referred to as a fixing support tool) will be described with reference to the drawings. First, the balloon unit for uterine hemostasis supported by the fixing support tool will be described.

### [Schematic Configuration of Balloon Unit for Uterine Hemostasis]

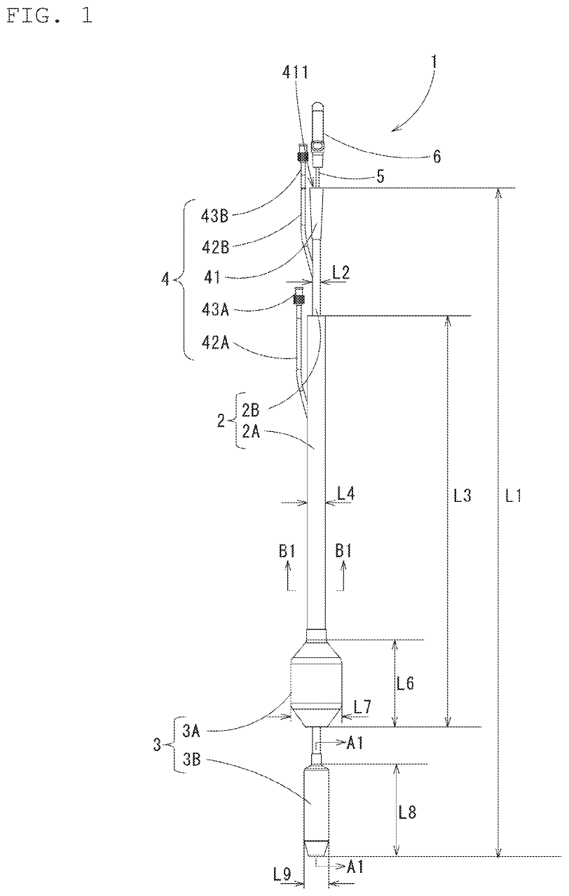

As illustrated in FIG. 1, a balloon unit for uterine hemostasis (hereinafter, referred to as a balloon unit) 1 according to the present embodiment includes a flexible tube 2, a hemostatic balloon 3 provided in a distal end portion of the tube 2, and a connector 4 provided in the tube 2.

### [Configuration of Tube]

The tube 2 has a first tube 2A and a second tube 2B. For example, each of the tubes 2A and 2B is formed of a synthetic resin such as polyvinyl chloride, silicone rubber, and a thermoplastic elastomer, and is flexible. A line (not illustrated) is formed in each of the tubes 2A and 2B along an extending direction of each of the tubes 2A and 2B. The line is formed in such a way that a material which can be detected by an X-ray is applied to or mixed with each of the tubes 2A and 2B. Out of these, the second tube 2B is formed to have a length approximately twice (1.8 to 2.2 times) that of the first tube 2A. Specifically, a length L3 of the first tube 2A is set to 360 mm to 400 mm, a length L1 of the second tube 2B is set to 660 mm to 880 mm, and a ratio between L3 and L1 is L3:L1=1:1.8 to 2.2. In addition, a diameter of the second tube 2B is formed to be smaller than a diameter of the first tube 2A, and thus, the second tube 2B can be inserted into the first tube 2A. Each of the first tube 2A and the second tube 2B will be described in detail below.

### [Configuration of Second Tube]

As illustrated in FIG. 1, a second balloon 3B is provided in a distal end portion of the second tube 2B, and a drain terminal 41 is connected to a proximal end portion of the second tube 2B. The second tube 2B penetrates the second balloon 3B, and membranes forming the second balloon 3B are fixed in an airtight manner at two locations including a distal end of the second tube 2B and a position away from the distal end by 80 mm to 120 mm. That is, the distal end of the second balloon 3B and the distal end of the second tube 2B coincide with each other. The distal end of the second tube 2B is open at a distal end position of the second balloon 3B.

The length L1 of the second tube 2B (length from a distal end of the second tube 2B to a proximal end portion of the drain terminal 41) is set to 660 mm to 880 mm. In addition, as illustrated in FIG. 5, an outer diameter L2 of the second tube 2B is set to approximately half (0.4 to 0.6 times) an inner diameter L5 (16 mm to 20 mm) of a first drain flow path 21A for discharging blood flowing into the uterus from the distal end to the outside. Specifically, the outer diameter L2 of the second tube 2B is set to 6.4 mm to 12 mm. In other words, a ratio between L5 and L2 is L5:L2=1:0.4 to 0.6. That is, the outer diameter L2 of the second tube 2B is set to a diameter that can be inserted into the first drain flow path 21A of the first tube 2A.

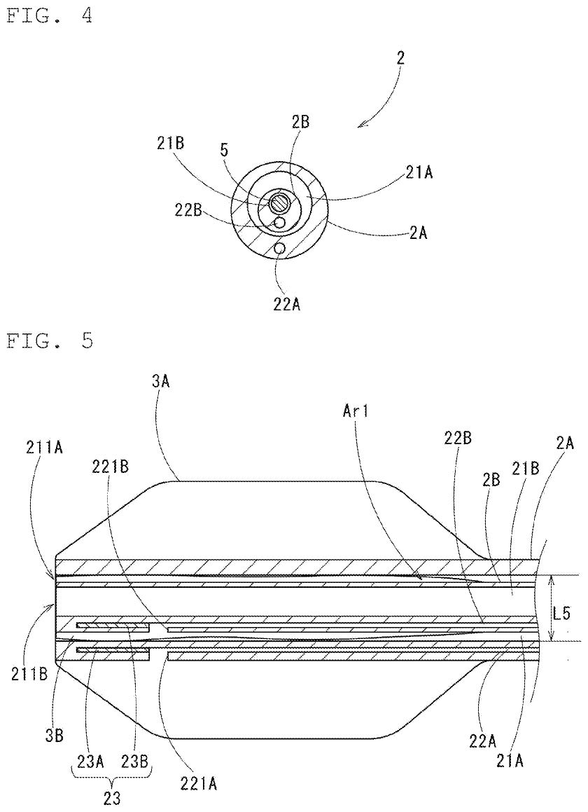

In addition, as illustrated in FIG. 3, the second tube 2B includes a second drain flow path 21B for discharging the blood flowing into the uterus from the distal end to the outside, and a second supply-discharge flow path 22B communicating with the second balloon 3B. An inner diameter of the second drain flow path 21B is set to 4.2 mm to 5.2 mm, and an inner diameter of the second supply-discharge flow path 22B is set to 1.1 mm to 2.6 mm. Out of these, the second drain flow path 21B communicates with a portion from the distal end to the proximal end of the second tube 2B. As illustrated in FIG. 3, a distal end portion thereof has an opening 211B into which the blood inside the uterus flows. On the other hand, the second supply-discharge flow path 22B communicates with a portion from the vicinity of the distal end to the vicinity of the proximal end of the second tube 2B. An opening 221B is formed on a side surface of the distal end portion of the second supply-discharge flow path 22B (side portion of a portion penetrating the second balloon 3B in the second tube 2B).

Here, the second tube 2B is provided with the line formed in such a way that the material which can be detected by the X-ray is applied thereto or mixed therewith as described above. Accordingly, although the position of the second tube 2B can be detected by the X-ray, when there is no X-ray inspection device, the position cannot be detected even when the second tube 2B of the balloon unit 1 is inserted into the uterus. In order to cope with this situation, in the present embodiment, as illustrated in FIG. 3, a detection member (hereinafter, referred to as a second detection member) 23B which can be detected by an ultrasonic echo is sealed on the distal end side of the second supply-discharge flow path 22B in the second tube 2B. The second detection member 23B is formed of a metal wire rod which can be detected by the ultrasonic echo. Therefore, even when there is no X-ray inspection device, the position of the second balloon 3B can be easily detected by the ultrasonic echo.

The detection member 23 (second detection member 23B and first detection member 23A (to be described later)) is formed of the metal which can be detected by the ultrasonic echo. However, preferably, the detection member 23 may be formed of a non-ferrous metal or stainless steel which is resistant to rust in the metal. More preferably, the detection member 23 is formed of aluminum which less affects a living body. In this case, the detection member 23 may be formed of pure aluminum having the same composition as that of a stylet 5 (to be described later).

### [Configuration of First Tube]

As illustrated in FIG. 1, the first balloon 3A is provided in the distal end portion of the first tube 2A. The first tube 2A penetrates the first balloon 3A, and the membranes forming the first balloon 3A are fixed in an airtight manner at two locations including the distal end of the first tube 2A and a position away from the distal end by 75 mm to 95 mm. That is, the distal end of the first balloon 3A and the distal end of the first tube 2A coincide with each other.

The length L3 of the first tube 2A is set to 360 mm to 400 mm, and the outer diameter L4 is set to 20 mm to 26 mm. As illustrated in FIG. 5, the first tube 2A has a first drain flow path 21A for discharging the blood flowing into the uterus from the distal end to the outside, and a first supply-discharge flow path 22A communicating with the first balloon 3A. The inner diameter L5 of the first drain flow path 21A is set to 16 mm to 20 mm, and the inner diameter of the first supply-discharge flow path 22A is set to 1.1 mm to 2.6 mm. Out of these, the first drain flow path 21A communicates with a portion from the distal end to the proximal end of the first tube 2A. As illustrated in FIG. 5, the distal end portion has an opening 211A through which the blood inside the uterus flows, and which is formed outward of the first balloon 3A at the distal end position of the first balloon 3A. On the other hand, the first supply-discharge flow path 22A communicates with a portion from the vicinity of the distal end to the vicinity of the proximal end of the first tube 2A. The distal end of the first supply-discharge flow path 22A is closed, and a side surface of the distal end portion (side portion of the portion penetrating the first balloon 3A in the first tube 2A) has an opening 221A formed inward of the first balloon 3A.

As illustrated in FIG. 5, in the first drain flow path 21A of the first tube 2A, an accommodation portion Ar1 for accommodating the second balloon 3B in a deflated state is continuously formed in the opening portion 211A. In addition, the first drain flow path 21A is formed so that the inner diameter L5 of the portion other than the accommodation portion Ar1 enables at least the second tube 2B to be inserted. Specifically, the inner diameter L5 (16 mm to 20 mm) of the first drain flow path 21A is set to be approximately twice the outer diameter L2 (6.4 mm to 12 mm) of the second tube 2B. In other words, a ratio between L5 and L2 is L5:L2=1:0.4 to 0.6. In the present embodiment, the first drain flow path 21A has a shape extending straight, and the inner diameter L5 is set so that the second balloon 3B in a deflated state and the tube 2B can be inserted. In addition, the second balloon 3B is slidable between an accommodation position accommodated in the accommodation portion Ar1 and a projecting position (position illustrated in FIG. 1) projecting from the accommodation portion Ar1.

In the present embodiment, the first drain flow path 21A extends straight, and the inner diameter L5 has the same shape at any position. However, the present invention is not limited thereto. For example, the diameter of the portion serving as the accommodation portion Ar1 may be set to be larger than the inner diameter L5 of the portion other than the accommodation portion Ar1, and a gap may be increased between the accommodation portion Ar1 and the second balloon 3B. In this case, the second balloon 3B can be smoothly moved in the accommodation portion Ar1.

In addition, in the first tube 2A, the first detection member 23A similar to the second detection member 23B is sealed on the distal end side of the first supply-discharge flow path 22A. Therefore, even when there is no X-ray inspection device, the position of the first balloon 3A can be easily detected by the ultrasonic echo.

### [Configuration of Hemostatic Balloon]

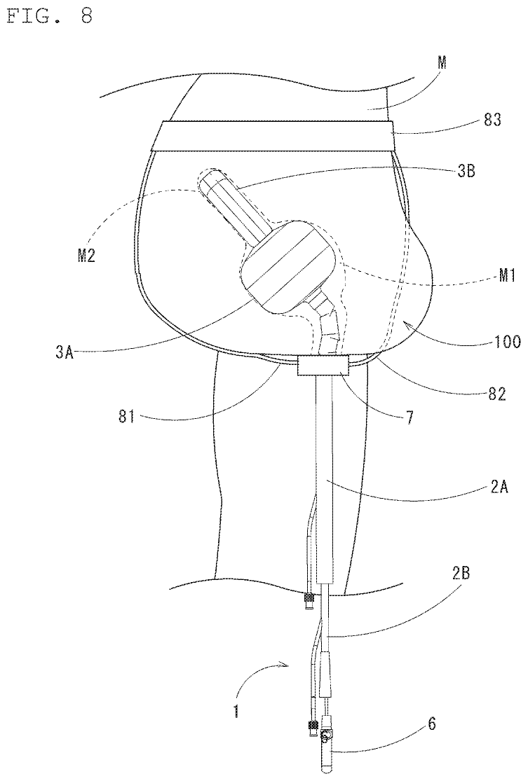

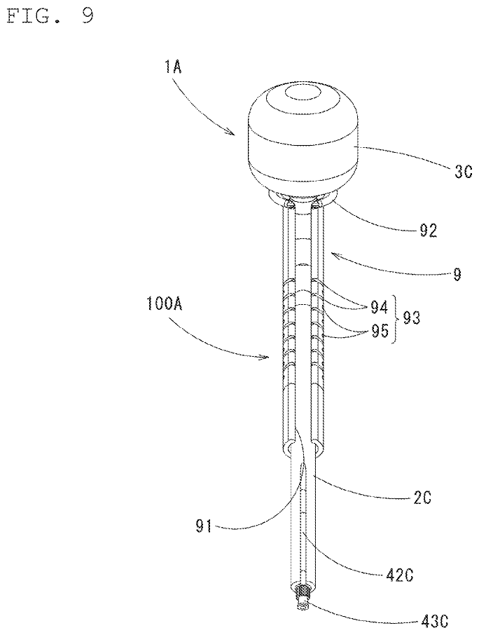

For example, the hemostatic balloon 3 is formed of silicone rubber or the like. The hemostatic balloon 3 has a first balloon 3A communicating with the first supply-discharge flow path 22A and a second balloon 3B communicating with the second supply-discharge flow path 22B, and is inflated by the liquid such as water or the like injected via each of the supply-discharge flow paths 22A and 22B. The first balloon 3A and the second balloon 3B are elastic and slightly inflated in a free state. As illustrated in FIG. 8, in an inflated state, the first balloon 3A is formed so that the length is shorter and the diameter is larger than those of the second balloon 3B. In other words, in the inflated state, the second balloon 3B is formed so that length is longer and the diameter is smaller than those of the first balloon 3A.

Out of these, the first balloon 3A is configured so that the liquid having a volume approximately twice (1.8 to 2.2 times) that of the second balloon 3B can be injected. For example, the liquid of maximum 450 ml to 880 ml can be injected. On the other hand, the second balloon 3B is configured so that the liquid of maximum 250 ml to 400 ml can be injected. A ratio between maximum capacity of the first balloon 3A and maximum capacity of the second balloon 3B is the maximum capacity of the first balloon 3A: the maximum capacity of the second balloon 3B=1.8 to 2.2:1. The maximum capacity of the liquid can be injected into each of the balloons 3A and 3B. However, when the balloon is inserted into the uterus, a proper amount according to a uterine shape is injected. More specifically, a proper amount according to a shape of a lower uterine lumen M1 is injected into the first balloon 3A, and a proper amount according to a shape of an upper uterine lumen M2 is injected into the second balloon 3B.

In addition, a length L6 of the first balloon 3A in a state where the liquid is not injected is set to 75 mm to 95 mm, and a diameter (outer diameter) L7 is set to 35 mm to 45 mm. The length L6 of the first balloon 3A when the liquid of 500 ml is injected is set to 90 mm to 110 mm, and the diameter (outer diameter) L7 is set to 95 mm to 105 mm. When the liquid is injected into the first balloon 3A in this way, the first balloon 3A has a substantially spherical shape as illustrated in FIG. 8. The first balloon 3A is inflated inside the uterus to compress an inner surface of the lower uterine lumen M1.

On the other hand, the second balloon 3B is configured so that the liquid having approximately half the volume of the first balloon 3A can be injected. As described above, the liquid of maximum 250 ml to 400 ml can be injected. Specifically, a length L8 in a state where the second balloon 3B is slightly inflated due to elasticity (deflated state illustrated in FIG. 5, more specifically, a state illustrated in FIGS. 1 and 3 instead of a state where the second balloon 3B is squeezed and accommodated in the accommodation portion Ar1 to be wrapped around the outer periphery of the second tube 2B) is set to 80 mm to 110 mm. A diameter (outer diameter) L9 is set to 19 mm to 29 mm. The length L8 when the liquid of 300 ml is injected is set to 100 mm to 130 mm. The diameter (outer diameter) L9 is set to 65 mm to 75 mm. When the liquid is injected into the second balloon 3B in this way, the second balloon 3B has a substantially spherical shape. However, as illustrated in FIG. 8, when accommodated in the upper uterine lumen M2, the second balloon 3B has a flat shape according to a shape thereof. The second balloon 3B is inflated inside the uterus. In this manner, as illustrated in FIG. 8, the second balloon 3B is located on an inner side from the lower uterine lumen M1, and compresses the inner surface of the upper uterine lumen M2 having the diameter smaller than that of the lower uterine lumen M1. A size (maximum capacity and various diameters) of each of the balloons 3A and 3B can be set in any desired way.

### [Configuration of Connector]

The connector 4 includes the drain terminal 41 communicating with the second drain flow path 21B and provided in the proximal end portion of the second tube 2B, the flexible second supply-discharge tube 42B communicating with the second supply-discharge flow path 22B on the distal end side from the drain terminal 41 and extending by branching outward of the second tube 2B, the supply-discharge terminal 43B provided in the proximal end of the second supply-discharge tube 42B and communicating with the second supply-discharge tube 42B, the flexible first supply-discharge tube 42A communicating with the first supply-discharge flow path 22A and extending by branching outward of the first tube 2A, and the supply-discharge terminal 43A provided in the proximal end of the first supply-discharge tube 42A and communicating with the first supply-discharge tube 42A.

The drain terminal 41 is a flexible tubular member formed of silicone rubber or the like. The drain terminal 41 is formed in a so-called trumpet shape whose diameter gradually increases toward the proximal end side. The drain terminal 41 communicates with the second drain flow path 21B. Accordingly, the blood flowing into the uterus from the opening 211B is discharged from an opening 411 of the drain terminal 41 via the second drain flow path 21B. A stylet 5 is inserted into the opening 411 of the drain terminal 41, and a cap 6 connected to the stylet 5 is attached to the drain terminal 41.

For example, each of the supply-discharge tubes 42A and 42B is formed of silicone rubber or the like, and is flexible. Each distal end of the supply-discharge tubes 42A and 42B communicate with each of the supply-discharge flow paths 22A and 22B inside each of the tubes 2A and 2B, and extends outward from the vicinity of the proximal end portions of each of the tubes 2A and 2B. Each of the supply-discharge terminals 43A and 43B is provided in each proximal end of the supply-discharge tubes 42A and 42B. For example, each of the supply-discharge terminals 43A and 43B is formed of hard polyvinyl chloride (PVC) or the like. A two-way cock (not illustrated) is connected to each of the supply-discharge terminals 43A and 43B.

A liquid dispenser (not illustrated) such as a syringe or the like is connected to the above-described two-way cock. When the liquid is injected from the liquid dispenser via the two-way cock, the liquid is supplied into each of the balloons 3A and 3B via the supply-discharge terminals 43A and 43B, the supply-discharge tubes 42A and 42B, and the supply-discharge flow paths 22A and 22B. On the other hand, when the two-way cock is unlocked in a state where the liquid is supplied into each of the balloons 3A and 3B, the liquid inside each of the balloons 3A and 3B flows back to deflate each of the balloons 3A and 3B.

### [Configuration of Stylet and Cap]

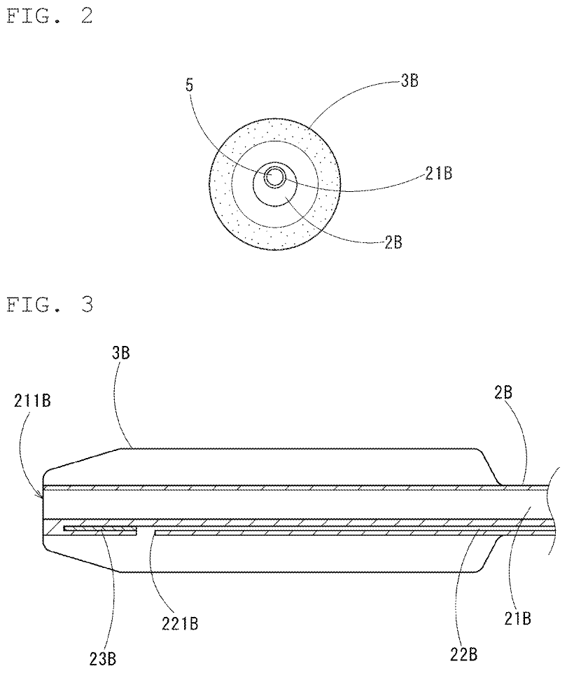

The stylet 5 is disposed inside the second drain flow path 21B as illustrated in FIGS. 2 and 4. The cap 6 detachably fitted to the opening 411 of the drain terminal 41 is fixed to the proximal end portion of the stylet 5. The cap 6 functions as a holder for putting a finger on the stylet 5 when operated by a medical worker. Out of these, the stylet 5 is formed of a wire of stainless steel, polypropylene, or the like, in addition to pure aluminum or an aluminum alloy (for example, A1070, A1080, or similar). In this manner, the stylet 5 can maintain a shape thereof at a bent angle of the stylet 5, and deforms when a strong force is applied thereto.

In the present embodiment, the length of the stylet 5 is set to the length in which the stylet 5 is located from a position where the distal end of the stylet 5 cannot project from the distal end of the second balloon 3B to the proximal end portion of the second balloon 3B, when the cap 6 is attached to the opening 411 of the drain terminal 41. In this manner, the balloon unit 1 can be properly inserted into the mother's body, and the stylet 5 is prevented from damaging the uterus.

When the distal end of the stylet 5 extends to the vicinity of the distal end of the second drain flow path 21B beyond a position which cannot project from the distal end of the second balloon 3B, there is a possibility that the stylet 5 may protrude from the distal end of the second drain flow path 21B. When the distal end of the stylet 5 does not extend to the proximal end portion of the second balloon 3B, the second balloon 3B cannot be supported by the stylet 5. Accordingly, the balloon unit 1 is less likely to be inserted into the mother's body.

### [Configuration of Fixing Support Tool]

FIG. 6 is a view illustrating a state where the balloon unit 1 disposed in the uterus is supported by a fixing support tool 100.

As illustrated in FIG. 6, the fixing support tool 100 fixes the balloon unit 1 to the patient (mother's body M). The fixing support tool 100 has a tube stopper 7 that supports the tube 2A of the balloon unit 1 and a fixing member 8 that can fix the tube stopper 7 to the patient.

### [Configuration of Tube Stopper]

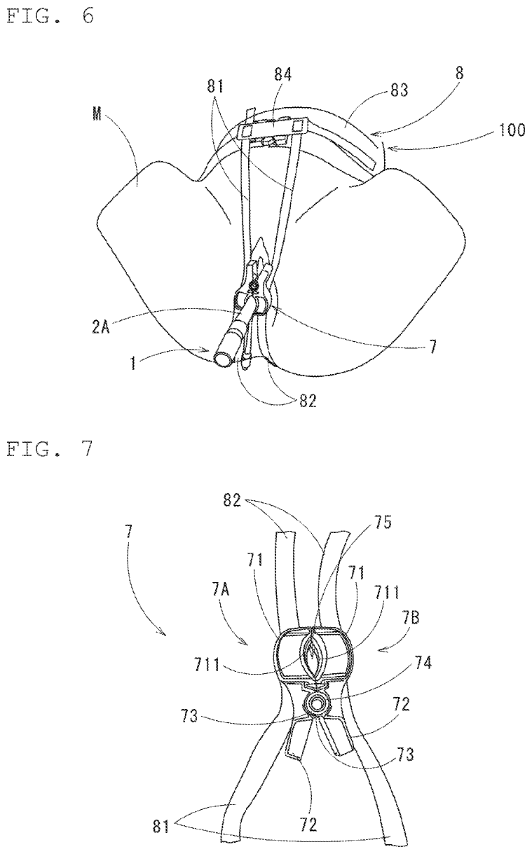

As illustrated in FIG. 6, the tube stopper 7 pinches and supports the tube 2A. As illustrated in FIG. 7, the tube stopper 7 includes a pair of pinching pieces 7A and 7B. Each of the pair of pinching pieces 7A and 7B includes a pinching portion 71 having a substantially semicircular arc shape in a plan view, a gripping portion 72 located on a side opposite to the pinching portion 71, and an intermediate portion 73 located between the pinching portion 71 and the gripping portion 72, and having a circular shape in a plan view. Both the pinching pieces 7A and 7B are integrated to be pivotable around on a shaft portion 74 by the shaft portion 74 that pivotally supports a hole portion formed in the center of the intermediate portion 73. Both the pinching pieces 7A and 7B can be opened and closed in a direction in which the pinching portions 71 are away from and closer to each other. In addition, the gripping portion 72 is provided with a spring that presses the pinching portion 71 in a direction in which distal ends of the pinching portions 71 come into contact with each other.

When the pinching portions 71 of each of the pinching pieces 7A and 7B are in contact with each other, the two semicircular arc-shaped pinching portions 71 are combined to form a space 75 having a substantially circular shape in a plan view between the pinching portions 71. The space 75 is formed to pinch the tube 2A, and an opening diameter of the space 75 is set to be smaller than the outer diameter of the tube 2A. For example, the opening diameter is set to 10 mm to 35 mm.

In addition, an elastic member 711 is fixed to the inner peripheral surface of each of the pinching portions 71. In this manner, when the tube 2A is pinched, the tube stopper 7 is prevented from being detached from the tube 2A or being deviated along the tube 2A.

Therefore, when each of the gripping portions 72 of the tube stopper 7 is gripped and moved in a direction in which both are closer to each other, the pinching portions 71 are moved in a direction in which both are away from each other, thereby forming a gap between the distal ends of the pinching portions 71. The diameter of the gap is set to be the same as the outer diameter of the tube 2A or to be slightly smaller than the outer diameter of the tube 2A. In this manner, the gripping portion 72 can be fitted into the tube 2A via the gap. When a hand is released from each of the gripping portions 72, the pinching portions 71 are moved in a direction in which both are closer to each other by the spring. Accordingly, the tube 2A can be gripped.

### [Configuration of Fixing Member]

The fixing member 8 includes bands 81 and 82 connected to the tube stopper 7, and an abdominal belt 83 wrapped around and attached to an abdomen of a patient (mother's body M). The band 81 is fixed to the proximal end portion of the pinching portion 71 of the pinching pieces 7A and 7B, and the band 82 is fixed to the distal end portion of the pinching portion 71. The band 82 is connected to a back side of the abdominal belt 83, and the band 81 is connected to a ventral side of the abdominal belt 83. For example, the bands 81 and 82 are formed of a vinyl chloride resin, a silicone resin, or the like, and the length is fixed to be adjustable by an adjustment portion 84 of the abdominal belt 83. When the bands 81 and 82 are connected to the adjustment portion 84 of the abdominal belt 83 and are pulled toward the abdominal belt 83 side, the tube stopper 7 to which the bands 81 and 82 are connected is pulled toward a crotch of the mother's body M. Front surfaces of both the pinching pieces 7A and 7B of the tube stopper 7 come into contact with the crotch (vulva) of the mother's body M.

### [Method of Using Balloon Unit]

FIG. 8 is a view illustrating a balloon unit 1 disposed in the uterus and supported by the fixing support tool 100 in a state of compressing the lower uterine lumen M1 and the upper uterine lumen M2. In FIG. 8, in order to facilitate understanding each configuration, there are gaps between the first balloon 3A and the inner surface of the lower uterine lumen M1 and between the second balloon 3B and the inner surface of the upper uterine lumen M2. However, actually, each of the balloons 3A and 3B compresses each of the above-described inner surfaces without any gap.

First, the balloon unit 1 is gripped, and the tubes 2A and 2B are bent at a desired angle. Thereafter, as illustrated in FIG. 8, in a state where the second balloon 3B is accommodated in the accommodation portion Ar1 of the first tube 2A, the first balloon 3A is inserted into the vagina, and is inserted into the uterus (lower uterine lumen M1) via the uterine os.

Then, the liquid is caused to flow only into the first balloon 3A via the first supply-discharge tube 42A and the first supply-discharge flow path 22A. In this manner, the inner surface of the lower uterine lumen M1 is in a state of being compressed by the first balloon 3A.

In addition, the inner surface of the lower uterine lumen M1 is compressed by the first balloon 3A. Thereafter, the cap 6 connected to the stylet 5 is gripped, and is pushed inward. In this manner, the second balloon 3B slides inside the first drain flow path 21A, and projects to the inner side from the first balloon 3A. Then, the second balloon 3B reaches the upper uterine lumen M2. Thereafter, the liquid is caused to flow into the second balloon 3B via the second supply-discharge tube 42B and the second supply-discharge flow path 22B. In this manner, the inner surface of the upper uterine lumen M2 is in a state of being compressed by the second balloon 3B (state illustrated in FIG. 8).

Then, it is confirmed whether or not hemostasis can be performed by the compression of each of the balloons 3A and 3B. When it is determined that the hemostasis can be performed, the abdominal belt 83 is wrapped around and attached to the mother's body M. Then, the band 82 fixed to the distal end portion of the pinching portion 71 of the tube stopper 7 is fixed to a back side portion of the abdominal belt 83. Then, when the gripping portions 72 of the tube stopper 7 are gripped by the first tube 2A and the gripping portions 72 are brought close to each other, the pinching portions 71 move in a direction away from each other, and the gap is formed between the distal ends of the pinching portions 71. The pinching portion 71 of the tube stopper 7 is fitted into the tube 2A via the gap. In this case, it is preferable that the tube stopper 7 pinches a portion of the first tube 2A projecting from the vagina of the mother's body M, which is located at a position of 0 mm from the vagina. That is, it is preferable that the tube stopper 7 is in close contact with the vulva of the mother's body M. In this manner, it is possible to prevent the positioned first balloon 3A and second balloon 3B from being deviated inside the uterus.

Then, the band 81 on the distal end side of the pinching portion 71 of the tube stopper 7 is fixed to the adjustment portion 84 of the abdominal belt 83, and the length of the band 81 is adjusted. In this manner, the bands 81 and 82 are in a state of pulling the tube stopper 7 toward the abdominal belt 83, and are fixed in a state where the front surfaces of both the pinching pieces 7A and 7B of the tube stopper 7 are in contact with the crotch of the mother's body M. As a result, the balloon unit 1 is supported by the fixing support tool 100 in a state where the inner surfaces of the lower uterine lumen M1 and the upper uterine lumen M2 are compressed by each of the balloons 3A and 3B.

In the present embodiment, the tube stopper 7 is fixed to the patient (mother's body M) by the fixing member 8. Accordingly, the first tube 2A supported by the tube stopper 7 is fixed to the patient. Therefore, it is possible to prevent each of the balloons 3A and 3B inflated inside the uterus from falling out of the uterus. In addition, the fixing member 8 is configured to include the abdominal belt 83 and the bands 81 and 82. Accordingly, the tube stopper 7 can be reliably fixed to the mother's body M.

In addition, the tube stopper 7 is fixed to the mother's body M in a state where the front surfaces of both the pinching pieces 7A and 7B are in contact with the crotch of the mother's body M. Accordingly, an unreasonable force generated by twisting or bending is not applied to the first tube 2A. The first tube 2A projecting from the vagina can be stably supported.

### Second Embodiment

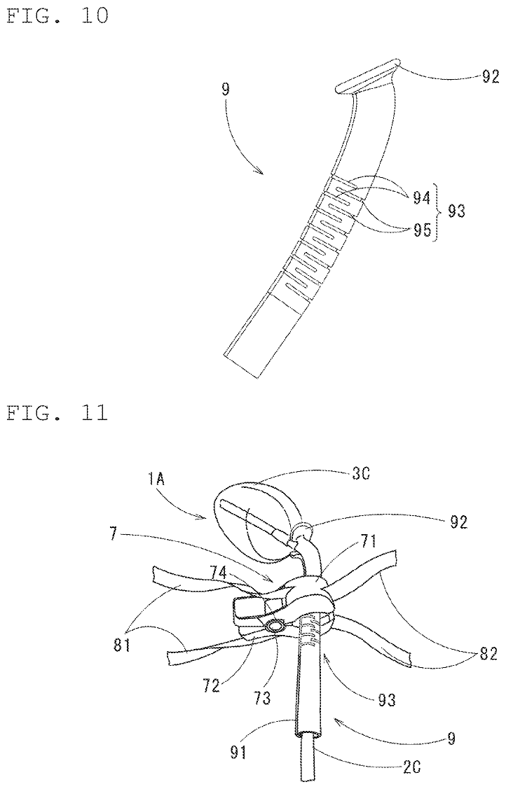

Next, a second embodiment of the present invention will be described with reference to the drawings. FIG. 9 is a schematic view illustrating a state where a tube cover 9 forming a fixing support tool 100A is attached to a balloon unit for uterine hemostasis 1A according to the present embodiment. The present embodiment is characterized in that the fixing support tool 100A has the tube cover 9, and other configurations are substantially the same as those of the first embodiment. Therefore, hereinafter, the same reference numerals will be assigned to configurations which are the same or substantially the same as those of the first embodiment, and description thereof will be omitted or simplified.

The balloon unit 1A of the present embodiment is different from the balloon unit 1 of the first embodiment, and includes one tube 2C and one hemostatic balloon 3C. The balloon unit 1A is a so-called single balloon unit. Accordingly, the outer diameter of the tube 2C is formed to be substantially the same as the outer diameter of the second tube 2B of the first embodiment. An internal structure thereof is the same as that of the second tube 2B. In addition, the hemostatic balloon 3C has a structure which is substantially the same as that of the first balloon 3A. The outer diameter of the tube 2C of the balloon unit 1A is as small as 8 mm to 12 mm. Accordingly, rigidity is relatively low (soft). Even when the tube 2C is pinched by the tube stopper 7, it is not easy to properly pinch and support the tube 2C. Therefore, in the present embodiment, the tube cover 9 is provided to be attached to the tube 2C to improve the rigidity.

### [Configuration of Tube Cover]

As illustrated in FIGS. 9 and 10, the tube cover 9 is formed in a tubular shape that covers the tube 2C. The inner diameter is set to be approximately the same as the outer diameter of the tube 2C, and is set to 8 mm to 12 mm, for example. For example, the tube cover 9 is formed of a resin such as silicone, vinyl chloride, or the like, and is formed to be elastically deformable. In addition, the tube cover 9 has a slit 91 linearly extending in the length direction of the tube cover 9, and used to attach the tube cover 9 to the tube 2C by opening the tube cover 9. That is, the slit 91 is configured to include one cutout extending in the length direction of the tube cover 9. A gap is formed by opening the tube cover 9 via the slit 91, and the tube cover 9 is attached to the tube 2C by laterally fitting the tube 2C into the gap.

In the present embodiment, for example, the width of the slit 91 is set to 5 mm to 10 mm. When the tube cover 9 is attached to the tube 2C, a portion of the tube 2C is visible. In this manner, the tube cover 9 can be easily attached to the tube 2C via the slit 91. In addition, although the tube cover 9 can cover the tube 2C, a region does not need to be a substantially entire region in the circumferential direction of the tube 2C. The tube cover 9 may cover a region which exceeds at least half in the circumferential direction of the tube 2C.

In addition, a cushion portion 92 is formed in an end portion on the hemostatic balloon 3C side of the tube cover 9 when the tube cover 9 is attached to the tube 2C. The cushion portion 92 is formed in a shape (so-called trumpet shape) whose outer diameter increases toward the end portion, and is formed to be thinner to be easily elastically deformed, compared to the region other than the cushion portion 92 in the tube cover 9. The cushion portion 92 comes into contact with the hemostatic balloon 3C when the tube cover 9 is attached to the tube 2C.

Furthermore, the tube cover 9 has a plurality of fixing portions 93 for fixing the tube stopper 7 at different positions in the length direction of the tube 2C. The fixing portion 93 includes a plurality of first recess portions 94 extending from the end portion of the slit 91 to an intermediate portion in the circumferential direction in a direction orthogonal to the length direction of the tube cover 9, and a plurality of second recess portions 95 formed between the adjacent first recess portions 94. That is, the plurality of first recess portion 94 are formed in approximately circumferential half on one side of the tube cover 9. The plurality of second recess portions 95 are formed in approximately circumferential half from a side opposite to the first recess portion 94 by 180°. The plurality of first recess portions 94 and second recess portions 95 are alternately disposed to intersect with the length direction of the tube cover 9. In addition, each of the first recess portions 94 and the second recess portions 95 has a shape recessed from the outer peripheral surface of the tube cover 9 toward the center of the tube cover 9, and is formed to be thinner than a region where the first recess portion 94 and the second recess portion 95 are not formed.

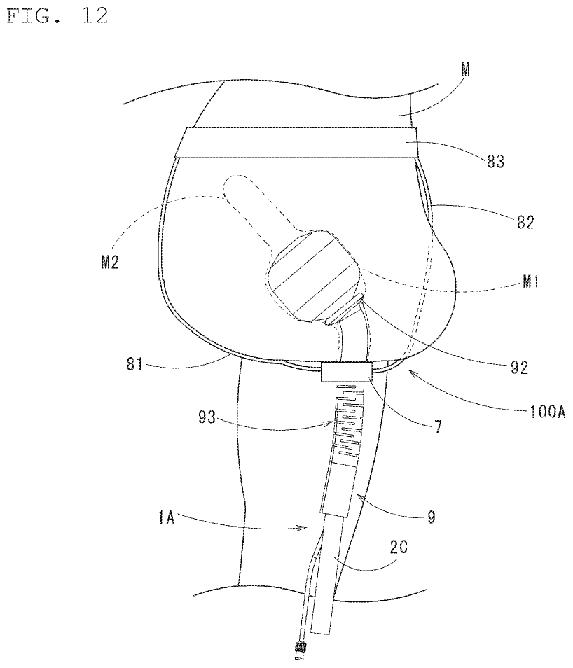

As illustrated in FIG. 11, the balloon unit 1A to which such the tube cover 9 is attached is pinched by the tube stopper 7 via the tube cover 9. In this case, it is preferable that a position pinched by the tube stopper 7 is a place where the fixing portion 93 of the tube cover 9 is formed. In this manner, the elastic member 711 formed on the outer peripheral surface on the space 75 side in each of the pinching portions 71 is locked to the fixing portion 93 (recess portions 94 and 95).

### [Method of Using Balloon Unit]

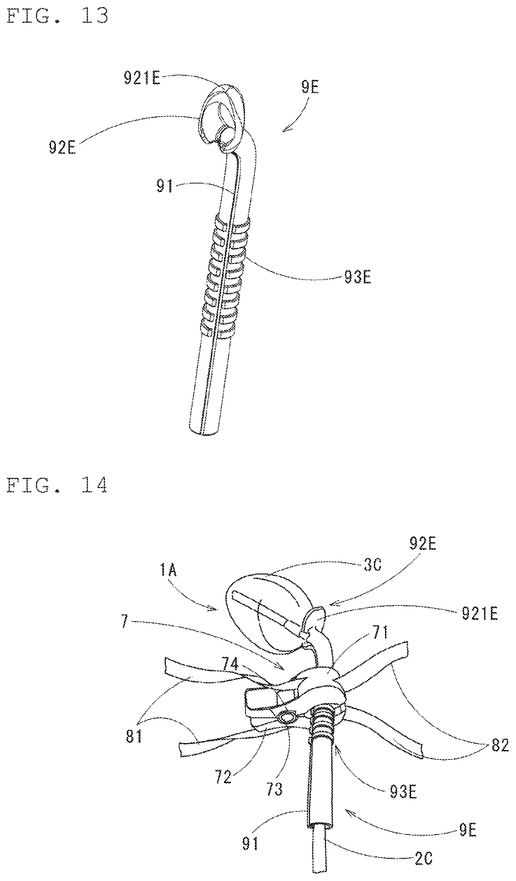

FIG. 12 is a view illustrating a balloon unit 1A in a state of being disposed inside the uterus and supported by the fixing support tool 100A in a state of compressing the lower uterine lumen M1. In FIG. 11, in order to facilitate understanding each configuration, there is a gap between the hemostatic balloon 3C and the inner surface of the lower uterine lumen M1. However, actually, the hemostatic balloon 3C compresses the above-described inner surface without any gap.

First, the tube stopper 7 is attached to the tube 2C via the slit 91. Thereafter, the balloon unit 1A is gripped and bent at a desired angle. Thereafter, the hemostatic balloon 3C and the tube 2C are inserted into the vagina as illustrated in FIG. 12 so that the hemostatic balloon 3C is inserted into the uterus (lower uterine lumen M1) via the uterine os. Then, the liquid is caused to flow into the hemostatic balloon 3C via the supply-discharge tube 42C. In this manner, the inner surface of the lower uterine lumen M1 is in a state of being compressed by the hemostatic balloon 3C.

Then, it is confirmed whether or not the hemostasis can be performed by the compression of the hemostatic balloon 3C. When it is determined that the hemostasis can be performed, the abdominal belt 83 is wrapped around and attached to the mother's body M. Then, the band 82 fixed to the distal end portion of the pinching portion 71 of the tube stopper 7 is fixed to a back side portion of the abdominal belt 83. Then, when the gripping portions 72 of the tube stopper 7 are gripped by the tube 2C and the gripping portions 72 are brought close to each other, the pinching portions 71 move in a direction away from each other, and the gap is formed between the distal ends of the pinching portions 71. The pinching portion 71 of the tube stopper 7 is fitted into the tube 2C having the attached tube cover 9 via the gap. In this case, it is preferable that the tube stopper 7 pinches a portion of the tube 2C projecting from the vagina of the mother's body M, which is located at a position of 0 mm from the vagina, that is, a portion where the fixing portion 93 of the tube cover 9 is formed. That is, it is preferable that the tube stopper 7 is in close contact with the vulva of the mother's body M. In this manner, it is possible to prevent the positioned hemostatic balloon 3C from being deviated inside the uterus.

Then, the band 81 on the pinching portion 71 side of the tube stopper 7 is fixed to the adjustment portion 84 of the abdominal belt 83, and the length of the band 81 is adjusted. In this manner, the bands 81 and 82 are in a state of pulling the tube stopper 7 toward the abdominal belt 83, and are fixed in a state where the front surfaces of the pinching portions 71 of both the pinching pieces 7A and 7B of the tube stopper 7 are in contact with the crotch of the mother's body M. As a result, the balloon unit 1A in a state where the inner surface of the lower uterine lumen M1 is compressed by the hemostatic balloon 3C is supported by the fixing support tool 100A.

In the present embodiment, the tube cover 9 is attached to the tube 2C. Accordingly, the rigidity of the tube 2C can be improved, and the tube 2C can be easily gripped and operated via the tube cover 9. In addition, even when the tube cover 9 is pushed toward the inflated hemostatic balloon 3C in a state where the tube cover 9 is attached to the tube 2C, the cushion portion 92 is provided in the distal end portion of the tube cover 9. Accordingly, the hemostatic balloon 3C can be prevented from being damaged by the tube cover 9. In addition, the tube 2C having the attached tube cover 9 is pinched by the tube stopper 7. Accordingly, the rigidity of the tube 2C (rigidity as a composite structure in which the tube 2C and the tube cover 9 are combined with each other) can be improved. In addition, the outer diameter of the composite structure in which the tube 2C and the tube cover 9 are combined with each other when the tube cover 9 is attached to the tube 2C is larger than the outer diameter of the tube 2C when the tube 2C is used alone. Accordingly, the tube 2C can be properly pinched and supported by the tube stopper 7. In addition, the tube cover 9 has the slit 91. Accordingly, the tube cover 9 can be easily attached to the tube 2C.

In addition, the tube cover 9 is provided with the plurality of fixing portions 93 (each of the recess portions 94 and 95). Accordingly, the tube stopper 7 can be fixed at a desired position in the length direction of the tube 2C. Moreover, the tube stopper 7 pinching the tube 2C is prevented from sliding and moving along the tube cover 9 even when an unexpected situation occurs. In this manner, a pinching position pinched by the tube stopper 7 can be maintained. In addition, the plurality of fixing portions 93 include the first recess portion 94 and the second recess portion 95 which are recessed inward. The plurality of fixing portions 93 do not project outward of the outer peripheral surface of the tube cover 9. Accordingly, when inserted into the vagina of the mother's body M, the tube 2C having the attached tube cover 9 can be smoothly inserted into the vagina, and the burden on the mother's body can be reduced.

The present invention is not limited to each of the above-described embodiments, and various modifications can be added within the scope not departing from the concept of the present invention.

For example, in each of the above-described embodiments, the fixing member 8 is configured to include the bands 81 and 82 and the abdominal belt 83. However, the present invention is not limited thereto. For example, the fixing member 8 may be configured so that a pants-type fixing member has an opening portion having a size through which the tube 2A can be inserted and the tube stopper 7 cannot be inserted. In this case, the balloon unit 1 in a state where the tube 2A is inserted into an opening portion of the pants-type fixing member may be inserted into the mother's body M, and the tube stopper 7 may be fixed to the tube 2A so that the pants-type fixing member is attached the mother's body M. Even in this case, the tube stopper 7 is fixed to the mother's body M. Accordingly, an advantageous effect the same as that of each of the above-described embodiments can be achieved.

For example, the pants-type fixing member may be formed of a water-absorbent material. In this case, the liquid such as the blood or the like flowing out via the balloon units 1 and 1A can be absorbed by the pants-type fixing member.

In addition, the fixing member 8 may be configured to include the bands 81 and 82. In this case, the bands 81 and 82 may be used as so-called suspenders, and may be hooked on both shoulders of the mother's body M so that the tube stopper 7 is fixed to the mother's body M. That is, the fixing member 8 may fix the tube stopper 7 to the mother's body M, regardless of the aspect of the above-described embodiments.

In the second embodiment, an example has been described in which the tube cover 9 is applied to the balloon unit 1A. However, the present invention is not limited thereto. For example, the tube cover 9 is also applicable to the balloon unit 1 illustrated in the first embodiment.

The fixing portion 93 of the second embodiment may be formed on the outer peripheral surface of the first tube 2A of the first embodiment.

In the second embodiment, as illustrated in FIG. 9, the slit 91 formed in the tube cover 9 has a shape linearly extending in the length direction of the tube cover 9. However, the present invention is not limited thereto. For example, the slit 91 may have a shape extending in a wavy line shape or a shape extending in a zigzag manner.

In the second embodiment, the fixing portion 93 of the tube cover 9 is configured to include the plurality of recess portions 94 and 95. However, the present invention is not limited thereto. The fixing portion 93 may be configured to include a plurality of projection portions. That is, as long as the tube stopper 7 can be locked, any configuration may be adopted. For example, a shape illustrated in FIG. 13 may be used.

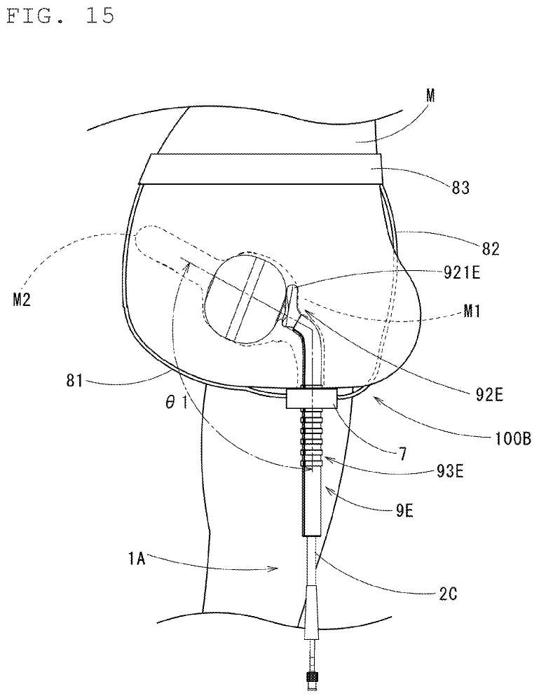

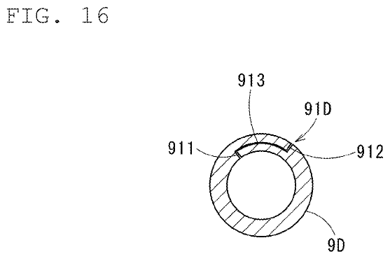

FIG. 13 is a perspective view illustrating a tube cover 9E according to a first modification example of the second embodiment. FIG. 14 is a perspective view illustrating a state where a tube 2C having the attached tube cover 9E is pinched by the tube stopper 7. FIG. 15 is a schematic view illustrating a state where the balloon unit 1A having the attached tube cover 9E is inserted into the mother's body M. In the following description, the same reference numerals will be assigned to configurations which are the same or substantially the same as those of the tube cover 9 of the second embodiment, and description thereof will be omitted or simplified.

As illustrated in FIGS. 13 to 15, the tube cover 9E according to the present modification example is formed in a tubular shape covering the tube 2C. The inner diameter is set to be approximately the same as the outer diameter of the tube 2C, and is set to 8 mm to 12 mm, for example. For example, the tube cover 9E is formed of a resin such as silicone, vinyl chloride, or the like, and is elastically deformable. In addition, the tube cover 9E linearly extends in the length direction of the tube cover 9E, and the distal end portion is bent. Specifically, as illustrated in FIG. 15, an angle θ1 formed by a linear portion of the tube cover 9E and the distal end portion side of the tube cover 9E is set to 100° to 170°. It is more preferable that the angle θ1 is 120° to 150°. The tube cover 9E has the slit 91 for attaching the tube cover 9E to the tube 2C by opening the tube cover 9E.

In addition, when the tube cover 9E is attached to the tube 2C, a cushion portion 92E is formed in an end portion on the hemostatic balloon 3C side of the tube cover 9E (end portion on a side that is bent inward). The cushion portion 92E is formed in a shape (so-called trumpet shape) whose outer diameter increases toward the end portion, and is formed to be thinner to be easily elastically deformed, compared to the region other than the cushion portion 92E in the tube cover 9E. In addition, the cushion portion 92E has an extension portion 921E extending substantially parallel to a linearly extending portion of the tube cover 9E. As illustrated in FIG. 15, the extension portion 921E is a portion that comes into contact with a portion in the vicinity of an entrance from the vagina to the lower uterine lumen M1 (portion on the back side of the lower uterine lumen M1) when the hemostatic balloon 3C is inflated inside the lower uterine lumen M1.

As described above, the distal end side of the tube cover 9E is bent inward. Accordingly, the rotation of the tube cover 9E in the vagina in the circumferential direction is prevented. In this manner, the extension portion 92E (extension portion 921E) of the tube cover 9E can reliably support the hemostatic balloon 3C by coming into contact with the portion on the back side of the lower uterine lumen M1. Therefore, for example, even when a doctor presses the abdomen of the mother's body M, the hemostatic balloon 3C can be prevented from escaping to the back side of the mother's body M. In this manner, a treatment the same as two-handed compression (hemostatic method in which the uterus and the abdomen are compressed by pressing hands) can be performed by using the doctor's hands and the hemostatic balloon 3C.

In addition, the tube cover 9E has a plurality of fixing portions 93E for fixing the tube stopper 7 at different positions in the length direction of the tube 2C. The fixing portion 93E is configured to include a plurality of projection portions extending from the end portion of the slit 91 in a direction orthogonal to the length direction of the tube cover 9. That is, the plurality of projection portions forming the fixing portion 93E project outward from the outer peripheral surface of the tube cover 9, and are formed to be thicker, compared to the region in which the fixing portion 93E is not formed. Therefore, the elastic member 711 formed on the outer peripheral surface on the space 75 side by each of the pinching portions 71 is reliably locked to the fixing portion 93E configured to include the plurality of projection portions. In addition, the rigidity of the tube cover 9E can be improved, compared to a case where the fixing portion 93 is configured to include the plurality of recess portions 94 and 95 as in the second embodiment.

In the first modification example of the second embodiment, when the slit 91 formed in the tube cover 9 is attached to the tube 2C, a portion of the tube 2C is visible from the outside. However, the present invention is not limited thereto. For example, a shape illustrated in FIG. 16 may be used.

As illustrated in FIG. 16, a slit 91D of a tube cover 9D includes a first slit 911 formed on an inner surface of the tube cover 9D and linearly extending in the length direction of the tube cover 9D, a linear second slit 912 formed at a position different from that of the first slit 911 on an outer surface of the tube cover 9D and extending in the length direction of the tube cover 9D, and a third slit 913 connecting the first slit 911 and the second slit 912 to each other. The first slit 911 and the second slit 912 are formed to have approximately half the depth in the radial direction from each of the inner surface and the outer surface of the tube cover 9D, and the first slit 911 and the second slit 912 are connected by the third slit 913 along the circumferential direction.

In this way, in the tube cover 9D, the first slit 911 and the second slit 912 are formed at positions where both of these do not overlap each other in the circumferential direction. Therefore, even when the tube cover 9D is attached to the tube 2C, the tube 2C is not in a visible state from the outside. Therefore, the tube 2C can be reliably covered by the tube cover 9D. Accordingly, a frictional force between the tube stopper 7 and the tube cover 9D increases. When the balloon unit 1 is inserted into the vagina of the mother's body M, the tube cover 9D moves along the tube 2C. In this manner, the hemostatic balloon 3C can be prevented from being pressed by the distal end of the tube cover 9D. In addition, the tube 2C is reliably covered by the tube cover 9D. Accordingly, the outer surface of the tube cover 9D can be smoothly formed. Therefore, the tube 2C can be easily inserted into the vagina.

Furthermore, in a state where the inflated hemostatic balloon 3C is in contact with the uterus, in a case where the tube cover 9D needs to be further pushed, when the tube 2C is covered as illustrated in FIG. 16, the tube 2C can be prevented from coming out from the tube cover 9D.

In the second embodiment, the tube covers 9, 9D, and 9E have the slits 91 and 91D. However, the present invention is not limited thereto. The slits 91 and 91D may not be provided. In this case, the tube covers 9, 9D, and 9E may be pushed and attached from the proximal end portion of the tube 2C.

In each of the above-described embodiments, the tube stopper 7 has a configuration in which the pinching pieces 7A and 7B including the pinching portion 71, the gripping portion 72, and the intermediate portion 73 are integrated with each other by the shaft portion 74. However, the present invention is limited thereto. For example, the tube stopper may be configured to have an opening portion into which the tubes 2A and 2C can be inserted, and may be configured so that the tube stopper is movable along the tubes 2A and 2C and can be fixed at a desired position. That is, the tube stopper 7 may have any desired configuration as long as the tube stopper 7 can support the first tube 2A and the tube 2C, and may not have a configuration for pinching each of the tubes 2A and 2C.

### INDUSTRIAL APPLICABILITY

The present invention can be used as the fixing support tool for fixing the balloon unit for uterine hemostasis to the patient.

### REFERENCE SIGNS LIST

1, 1A balloon unit for uterine hemostasis
2 tube
2A first tube
2B second tube
2C tube
21 drain flow path
21A first drain flow path
211A, 211B opening
21B second drain flow path
22 supply-discharge flow path
22A first supply-discharge flow path
221A opening
22B second supply-discharge flow path
221B opening
23 detection member
23A first detection member
23B second detection member
3 hemostatic balloon
3A first balloon
3B second balloon
3C hemostatic balloon
4 connector
41 drain terminal
411 opening
42 supply-discharge tube
42A first supply-discharge tube
42B second supply-discharge tube
43 supply-discharge terminal
43A first supply-discharge terminal
43B second supply-discharge terminal
5 stylet
6 cap
7 tube stopper
7A, 7B pinching piece
71 pinching portion
711 elastic member
72 gripping portion
73 intermediate portion
74 shaft portion
75 space
8 fixing member
81, 82 band
83 abdominal belt
84 adjustment portion
9, 9E tube cover
91, 91D slit
911 first slit
912 second slit
913 third slit
92 cushion portion
921E extension portion
93, 93E fixing portion
94 first recess portion
95 second recess portion
100, 100A fixing support tool
M mother's body (patient)
M1 lower uterine lumen
M2 upper uterine lumen

1. A fixing support tool of a balloon unit for uterine hemostasis, which is used to fix a balloon unit for uterine hemostasis including a flexible tube and a hemostatic balloon provided in a distal end of the tube to a patient, comprising:
a tube stopper that supports the tube; and
a fixing member capable of fixing the tube stopper to the patient.

2. The fixing support tool of a balloon unit for uterine hemostasis according to claim 1,
wherein the tube stopper has a pinching portion that pinches the tube.

3. The fixing support tool of a balloon unit for uterine hemostasis according to claim 1,
wherein the fixing member has an abdominal belt attachable to an abdomen of the patient, and a band connecting the abdominal belt and the tube stopper to each other.

4. The fixing support tool of a balloon unit for uterine hemostasis according to claim 1, further comprising:
a tubular tube cover that covers the tube,
wherein the tube stopper supports the tube via the tube cover.

5. The fixing support tool of a balloon unit for uterine hemostasis according to claim 4,
wherein the tube cover has a slit extending in a length direction of the tube and opening the tube cover so that the tube cover is attached to the tube.

6. The fixing support tool of a balloon unit for uterine hemostasis according to claim 4,
wherein the tube cover has a plurality of fixing portions for fixing the tube stopper at different positions in a length direction of the tube.

7. The fixing support tool of a balloon unit for uterine hemostasis according to claim 6,
wherein the fixing portion includes a recess portion recessed toward a center of the tube cover from an outer peripheral surface of the tube cover.

8. The fixing support tool of a balloon unit for uterine hemostasis according to claim 6,
wherein the fixing portion includes a projection portion projecting from an outer peripheral surface of the tube cover.

9. The fixing support tool of a balloon unit for uterine hemostasis according to claim 4,
wherein a cushion portion is formed in an end portion of the tube cover on the hemostatic balloon side when the tube cover is attached to the tube.
